(19) United States
(12) Patent Application Publication
(10) Pub. No.: US 2013/0096099 A1
(43) Pub. Date: Apr. 18, 2013

(54) METHOD OF TREATING BRAIN CANCER

(71) Applicant: Niiki Pharma Inc., Hoboken, NJ (US)

(72) Inventor: Hooshmand SHESHBARADARAN, Hoboken, NJ (US)

(73) Assignee: NIIKI PHARMA INC., Hoboken, NJ (US)

(21) Appl. No.: 13/663,438

(22) Filed: Oct. 29, 2012

Related U.S. Application Data
(63) Continuation of application No. PCT/US2011/034407, filed on Apr. 29, 2011.
(60) Provisional application No. 61/329,363, filed on Apr. 29, 2010.

(51) Int. Cl. A61K 31/555

(52) U.S. Cl. CPC A61K 31/555
USPC 514/186

(57) ABSTRACT

A therapeutic method for treating brain cancer is disclosed comprising administering to a patient in need of treatment a ruthenium complex salt.

### CROSS-REFERENCE TO RELATED U.S. APPLICATONS

This application is a continuation of PCT/US11/34407 filed on Apr. 29, 2011, which claims the benefit of U.S. Provisional Application No. 61/329,363 filed on Apr. 29, 2010, both of which are incorporated herein by reference.

### FIELD OF THE INVENTION

The present invention generally relates to methods for treating cancer, and particularly to a method of treating brain cancer.

### BACKGROUND OF THE INVENTION

It is estimated that there are over 40,000 new cases of brain cancer every year in the United States alone, and more than 13,000 die each year from the disease. Aside from surgery and radiation therapy, there are very few treatment options. Temozolomide and nitrosourea are the only accepted chemotherapeutics for brain cancer, and yet have shown rather limited effectiveness. Thus, there is a significant need for new agents in treating brain cancer.

Both Peti et al, Eur. J. Inorg. Chem. 1999, 1551-1555 and PCT Publication No. WO2008/154553 disclose methods of synthesizing the compound sodium trans-[tetrachlorobis(1H-indazole)ruthenate(III)].

### SUMMARY OF THE INVENTION

It has now been discovered that the compound sodium trans-[tetrachlorobis(1H-indazole)ruthenate(III)] is especially effective in inhibiting glioma cell growth. It has also been surprisingly discovered that the compound sodium trans-[tetrachlorobis(1H-indazole)ruthenate(III)] is equally effective in glioma cell lines both sensitive and resistant to drugs such as BCNU and temozolomide.

Accordingly, in a first aspect, the present invention provides a method of treating brain cancer, which comprises treating a patient identified as having brain cancer, with a therapeutically effective amount of trans-[tetrachlorobis(1H-indazole)ruthenate(III)] or a pharmaceutically acceptable salt thereof.

In a second aspect, the present invention provides a method of preventing or delaying the onset of brain cancer, comprising administering to a patient identified to be in need of prevention, or delaying the onset, of brain cancer a prophylatically effective amount of trans-[tetrachlorobis(1H-indazole)ruthenate(III)] or a pharmaceutically acceptable salt thereof.

The present invention further provides use of trans-[tetrachlorobis(1H-indazole)ruthenate(III)] or a pharmaceutically acceptable salt thereof for the manufacture of a medicament useful for treating, preventing or delaying the onset of brain cancer.

In yet another aspect, the present invention provides a method of treating refractory brain cancer comprising identifying a patient having refractory brain cancer and treating the patient with a therapeutically effective amount of trans-[tetrachlorobis(1H-indazole)ruthenate(III)] or a pharmaceutically acceptable salt thereof. In specific embodiments, the patient has a brain cancer that is refractory to a treatment comprising bis-chloronitrosourea (BCNU) and/or temozolomide.

The foregoing and other advantages and features of the invention, and the manner in which the same are accomplished, will become more readily apparent upon consideration of the following detailed description of the invention taken in conjunction with the accompanying examples, which illustrate preferred and exemplary embodiments.

### BRIEF DESCRIPTION OF THE DRAWINGS

FIG. 1 is a graph showing the dose-dependent growth inhibition by sodium trans-[tetrachlorobis(1H-indazole)ruthenate(III)] (MTT assay) in a 3 -dimentional tumor model (HuBiogel, Vivo Biosciences, Birmingham, Ala.) derived from glioma cell line U87.

### DETAILED DESCRIPTION OF THE INVENTION

The present invention is at least in part based on the discovery that the compound sodium trans-[tetrachlorobis(1H-indazole)ruthenate(III)] is effective in treating brain cancer. Accordingly, in accordance with a first aspect of the present invention, a method is provided for treating brain cancer. Specifically, the method comprises treating a patient having brain cancer with a therapeutically effective amount of trans-[tetrachlorobis(1H-indazole)ruthenate(III)] or a pharmaceutically acceptable salt thereof. That is, the present invention is directed to the use of trans-[tetrachlorobis(1H-indazole)ruthenate(III)] or a pharmaceutically acceptable salt thereof for the manufacture of medicaments for treating brain cancer in patients identified or diagnosed as having brain cancer.

The various aspects of the present invention can be useful in various brain malignancies including, but not limited to, acoustic neuroma, astrocytoma (e.g., pilocytic astrocytoma, low-grade astrocytoma, anaplastic astrocytoma), glioblastoma multiforme (GBM) and other gliomas (brain stem glioma, optic nerve glioma, ependymoma, mixed glioma, optic nerve glioma, oligodendroglioma, and subependymoma), chordoma, CNS lymphoma, craniopharyngioma, medulloblastoma, meningioma, primitive neuroectodermal (PNET), schwannoma, and rhabdoid tumor.

In the various embodiments of this aspect of the present invention, the treatment method optionally also comprises a step of diagnosing or identifying a patient as having brain cancer. The identified patient is then treated with or administered with a therapeutically effective amount of trans-[tetrachlorobis(1H-indazole)ruthenate(III)] or a pharmaceutically acceptable salt thereof (e.g., sodium trans-[tetrachlorobis(1H-indazole)ruthenate(III)] or indazolium trans-[tetrachlorobis(1H-indazole)ruthenate(III)]). Various brain cancers can be diagnosed in any conventional diagnostic methods known in the art including WI scan, CAT scan, PET scan, biopsy, etc.

In addition, it has also been surprisingly discovered that the compound sodium trans-[tetrachlorobis(1H-indazole)ruthenate(III)] is equally effective in brain cancer cells resistant to nitrosourea (e.g., BCNU) or temozolomide. Accordingly, the present invention also provides a method of treating refractory brain cancer comprising treating a patient identified as having refractory brain cancer with a therapeutically effective amount of trans-[tetrachlorobis(1H-indazole)ruthenate(III)] or pharmaceutically acceptable salt thereof (e.g., sodium trans-[tetrachlorobis(1 H-indazole)ruthenate(III)] or indazolium trans-[tetrachlorobis(1H-indazole)ruthenate(III)]). In one embodiment, the patient has a brain cancer that is refractory to a treatment comprising a nitrosourea drug such as BCNU. In another embodiment, the patient has a brain cancer that is refractory to a treatment comprising temozolomide. That is, the present invention is also directed to the use of trans-[tetrachlorobis(1H-indazole)ruthenate(III)] or a pharmaceutically acceptable salt thereof (e.g., sodium trans-[tetrachlorobis(1H-indazole)ruthenate(III)] or indazolium trans-[tetrachlorobis(1H-indazole)ruthenate(III)]) for the manufacture of medicaments for treating refractory brain cancer, e.g., a brain cancer refractory to nitrosourea or temozolomide.

The term “refractory brain cancer,” as used herein refers to brain cancer that either fails to respond favorably to an anti-neoplastic treatment that does not include trans-[tetrachlorobis(1H-indazole)ruthenate(III)], or alternatively, recurs or relapses after responding favorably to an antineoplastic treatment that does not include trans-[tetrachlorobis(1H-indazole)ruthenate(III)]. Accordingly, “a brain cancer refractory to a treatment” as used herein means a brain cancer that fails to respond favorably to, or resistant to, the treatment, or alternatively, recurs or relapses after responding favorably to the treatment.

Thus, in some embodiments, in the method of the present invention, trans-[tetrachlorobis(1H-indazole)ruthenate(III)] or a pharmaceutically acceptable salt thereof is used to treat brain cancer patients having a tumor that exhibits resistance to a treatment comprising one or more drugs such as nitrosourea (e.g., BCNU) and temozolomide. In other words, the method is used to treat a brain cancer patient having previously been treated with a treatment regimen that includes one or more drugs such as nitrosourea (e.g., BCNU) and temozolomide, and whose brain cancer was found to be non-responsive to the treatment regimen or have developed resistance to the treatment regimen. In other embodiments, the method is used to treat a brain cancer patient previously treated with a treatment comprising one or more drugs such as nitrosourea (e.g., BCNU) and temozolomide, but the brain cancer has recurred or relapsed, that is, a brain cancer patient who has previously been treated with one or more such drugs, and whose cancer was initially responsive to the previously administered one or more such drugs, but was subsequently found to have relapsed. In specific embodiments, sodium trans-[tetrachlorobis(1H-indazole)ruthenate(III)] is used to treat brain cancer patients previously treated with a nitrosourea, i.e., who have a tumor that exhibits resistance to, or relapsed after a treatment including a nitrosourea (e.g., BCNU).

In other specific embodiments, sodium trans-[tetrachlorobis(1H-indazole)ruthenate(III)] is used to treat brain cancer patients previously treated with temozolomide, i.e., who have a brain cancer that exhibits resistance to, or relapsed after a treatment including temozolomide.

To detect a refractory brain cancer, patients undergoing initial treatment can be carefully monitored for signs of resistance, non-responsiveness or recurring brain cancer. This can be accomplished by monitoring the patient's cancer's response to the initial treatment which, e.g., may include nitrosourea or temozolomide. The response, lack of response, or relapse of the cancer to the initial treatment can be determined by any suitable method practiced in the art. For example, this can be accomplished by the assessment of tumor size and number. An increase in tumor size or, alternatively, tumor number, indicates that the tumor is not responding to the chemotherapy, or that a relapse has occurred. The determination can be done according to the “RECIST” criteria as described in detail in Therasse et al, J. Natl. Cancer Inst. 92:205-216 (2000).

In accordance with yet another aspect of the present invention, a method is provided for preventing or delaying the onset of brain cancer, or preventing or delaying the recurrence of brain cancer, which comprises treating a patient in need of the prevention or delay with a prophylatically effective amount of trans-[tetrachlorobis(1H-indazole)ruthenate(III)] or a pharmaceutically acceptable salt thereof (e.g., sodium trans-[tetrachlorobis(1H-indazole)ruthenate(III)] or indozolium trans-[tetrachlorobis(1H-indazole)ruthenate(III)]).

For purposes of preventing or delaying the recurrence of brain cancer, brain cancer patients who have been treated and are in remission or in a stable or progression free state may be treated with a prophylatically effective amount of trans-[tetrachlorobis(1H-indazole)ruthenate(III)] or a pharmaceutically acceptable salt thereof (e.g., sodium trans-[tetrachlorobis(1H-indazole)ruthenate(III)] or indazolium trans-[tetrachlorobis(1H-indazole)ruthenate(III)]) to effectively prevent or delay the recurrence or relapse of brain cancer.

As used herein, the phrase “treating . . . with . . . ” or a paraphrase thereof means administering a compound to the patient or causing the formation of a compound inside the body of the patient.

In accordance with the method of the present invention, brain cancer can be treated with a therapeutically effective amount of trans-[tetrachlorobis(1H-indazole)ruthenate(III)] or a pharmaceutically acceptable salt thereof (e.g., sodium trans-[tetrachlorobis(1H-indazole)ruthenate(III)] or indazolium trans-[tetrachlorobis(1H-indazole)ruthenate(III)]) alone as a single agent, or alternatively in combination with one or more other anti-cancer agents. Example of pharmaceutically acceptable salts include alkali metal salts (e.g., sodium or potassium salt), indazolium salts, etc. An alkali metal salt, preferably sodium salt of trans-[tetrachlorobis(1H-indazole)ruthenate(III)] (i.e., sodium trans-[tetrachlorobis(1H-indazole)ruthenate(III)] or potassium trans-[tetrachlorobis(1H-indazole)ruthenate(III)]) is particularly useful.

Alkali metal salts of trans-[tetrachlorobis(1H-indazole)ruthenate(III)] can be made in any methods known in the art. For example, PCT Publication No. WO/2008/154553 discloses an efficient method of making sodium trans-[tetrachlorobis(1H-indazole)ruthenate(III)]. U.S. Pat. No. 7,338,946 discloses indazolium trans-[tetrachlorobis(1H-indazole)ruthenate(III)]) and a formulation containing the indazolium salt.

The pharmaceutical compounds such as a pharmaceutically acceptable salt of trans-[tetrachlorobis(1H-indazole)ruthenate(III)] (e.g., sodium trans-[tetrachlorobis(1H-indazole)ruthenate(III)], or indazolium trans-[tetrachlorobis(1H-indazole)ruthenate(III)]) can be administered through intravenous injection or any other suitable means at an amount of from 0.1 mg to 1000 mg per kg of body weight of the patient based on total body weight. The active ingredients may be administered at once, or may be divided into a number of smaller doses to be administered at predetermined intervals of time, e.g., once daily or once every two days. It should be understood that the dosage ranges set forth above are exemplary only and are not intended to limit the scope of this invention. The therapeutically effective amount of the active compound can vary with factors including, but not limited to, the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, and the like, as will be apparent to a skilled artisan. The amount of administration can be adjusted as the various factors change over time.

In accordance with the present invention, it is provided a use of trans-[tetrachlorobis(1H-indazole)ruth enate(III)] or a pharmaceutically acceptable salt thereof (e.g., an alkali metal salt of trans-[tetrachlorobis(1H-indazole)ruthenate(III)] such as sodium trans-[tetrachlorobis(1H-indazole)ruthenate(III)] or potassium trans-[tetrachlorobis(1H-indazole)ruthenate(III)], or indazolium trans-[tetrachlorobis(1H-indazole)ruthenate(III)]) for the manufacture of a medicament useful for treating brain cancer. The medicament can be, e.g., in an injectable form, e.g., suitable for intravenous, intradermal, or intramuscular administration. Injectable forms are generally known in the art, e.g., in buffered solution or suspension.

In accordance with another aspect of the present invention, a pharmaceutical kit is provided comprising in a container a unit dosage form of a compound containing trans-[tetrachlorobis(1H-indazole)ruthenate(III)], or a pharmaceutically acceptable salt thereof (e.g., an alkali metal salt of trans-[tetrachlorobis(1H-indazole)ruthenate(III)] such as sodium trans-[tetrachlorobis(1H-indazole)ruthenate(III)] or potassium trans-[tetrachlorobis(1H-indazole)ruthenate(III)], or indazolium trans-[tetrachlorobis(1H-indazole)ruthenate(III)], and optionally instructions for using the kit in the methods in accordance with the present invention, e.g., treating, preventing or delaying the onset of brain cancer, or preventing or delaying the recurrence of brain cancer, or treating refractory brain cancer. As will be apparent to a skilled artisan, the amount of a therapeutic compound in the unit dosage form is determined by the dosage to be used on a patient in the methods of the present invention. In the kit, a compound having trans-[tetrachlorobis(1H-indazole)ruthenate(III)] or a pharmaceutically acceptable salt thereof (e.g., an alkali metal salt of trans-[tetrachlorobis(1H-indazole)ruthenate(III)] such as sodium trans-[tetrachlorobis(1H-indazole)ruthenate(III)] or potassium trans-[tetrachlorobis(1H-indazole)ruthenate(III)], or indazolium trans-[tetrachlorobis(1H-indazole)ruthenate(III)]) can be in lyophilized form in an amount of, e.g., 25 mg, in an ampoule. In the clinic, the lyophilized form can be dissolved in a buffer and administered to a patient in need of the treatment in accordance with the present invention.

### EXAMPLE

The compound sodium trans-[tetrachlorobis(1H-indazole)ruthenate(III)] was tested in a 3-dimentional tumor model derived from glioma cell line U87. Specifically, cells were trypsinized, washed, counted by trypan blue exclusion. Tumor beads were then prepared by mixing 20,000 cells/10 μl of HuBiogel (4 mg/mL) (See U.S. patent application Ser. No. 10/546,506, which is incorporated herein by reference). The 3-D tumor beads were cultivated for 72 hours in multi-well plates with complete media (10% FBS) in a 37° C. incubator +5% CO₂. Mini-tumors were treated with various concentrations of the test compound sodium trans-[tetrachlorobis(1H-indazole)ruthenate(III)] in media (final 0.2-0.3% DMSO) or control (DMSO). Repeated drug treatment was done by removing the culture media and replacing with fresh media with drug compound or DMSO. On Day 3, MTT assay and live-cell staining with Calcein AM were performed (5 beads/assay set).

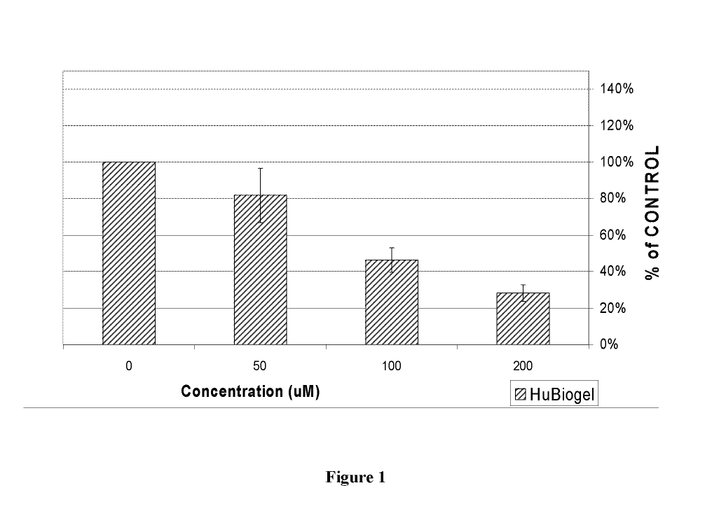

Sodium trans-[tetrachlorobis(1H-indazole)ruthenate(III)] exhibited dose-dependent tumor killing effective in live-cell staining/image analysis, and significantly inhibited tumor proliferation activity. See FIG. 1. Statistical analysis of data sets (Average, T-test, GI-50) was performed using MS-Excel program. The T-test result is shown in Table 1 below. The average GI-50 (the drug concentration required for growth inhibition at 50%) is 94.95 μM. Thus, sodium trans-[tetrachlorobis(1H-indazole)ruthenate(III)] is effective in U87 cell line which is relatively resistant to BCNU and temozolomide. See e.g., Bowles et al., J. Neurosurg., 73:248-253 (1990).

TABLE 1

| | Concentration (μM) | | |
|---|---|---|---|
| t-test | 200 | 100 | 50 |
| HuBiogel | 4.44798E−10 | 9.15941E−08 | 0.026 |
| (control vs | | | |
| experi- | | | |
| ment) | | | |
| | control vs 200 uM | control vs 100 uM | control vs 50 uM |

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be apparent that certain changes and modifications may be practiced within the scope of the appended claims.

What is claimed is:

1. A method of treating or delaying the onset of brain cancer comprising administering to a patient in need of treatment a pharmaceutically acceptable salt of trans-[tetrachlorobis(1H-indazole)ruthenate(III)].

2. The method of claim 1, wherein the brain cancer is a refractory brain cancer.

3. The method of claim 2, wherein said brain cancer is glioma.

4. The method of claim 2, wherein said brain cancer is astrocytoma.

5. The method according to claim 2, wherein said pharmaceutically acceptable salt is an alkali metal salt of trans-[tetrachlorobis(1H-indazole)ruthenate(III)]

6. The method according to claim 2, wherein said pharmaceutically acceptable salt is sodium trans-[tetrachlorobis(1H-indazole)ruthenate(III)].

7. The method of claim 2, wherein said brain cancer is refractory to a treatment comprising a nitrosourea or temozolomide.

8. The method of claim 7, wherein said brain cancer was previously treated with BCNU.

9. The method of claim 7, wherein said brain cancer was previously treated with temozolomide.
